(19) United States
(12) Patent Application Publication
(10) Pub. No.: US 2009/0306610 A1
(43) Pub. Date: Dec. 10, 2009

(54) Urine collecting device

(76) Inventors: Eleanor Van Den Heuvel, Slough (GB); Felicity Jowitt, Middlesex (GB)

Correspondence Address: Intellectual Property Dept.;Dewitt Ross & Stevens SC, 2 East Mifflin Street, Suite 600, Madison, WI, 53703-2865, US

(21) Appl. No.: 12/090,113

(22) PCT Filed: Oct. 3, 2006
(86) PCT No.: PCT/GB06/03680

§ 371 Date: Aug. 8, 2008

(30) Foreign Application Priority Data
Oct. 13, 2005 (GB) 0520863.2

(51) Int. Cl. A61M 27/00

(52) U.S. Cl. 604/317

(57) ABSTRACT

A urine collection device includes a membrane filter (9) through which urine flows prior to being collected in a storage container (1). In order to filter out particulate matter from the urine, and thus increase the lifespan of the membrane filter (9), a pre-filter (8) is provided upstream of the membrane filter (9). The pre-filter (8) has a higher capacity than the membrane filter (9). The membrane filter (9) and the pre-filter (8) may be provided within a single unit that may comprise the lid of the storage container (1). An air outlet (2) may include moisture reduction means, such as at least one hydrophobic membrane (3, 5) and/or moisture-absorbing material (4). Moisture-absorbing material (4) may also serve to absorb odours.

The present invention relates to a urine collection device.

Millions of people world wide are afflicted with the problem of urinary incontinence, the economic costs of this distressing condition are extremely high but the cost in terms of human suffering is incalculable. Both men and women are afflicted by the condition although many more women are affected and incidence increases with age.

For many younger men external catheter systems provide an adequate solution to the problem of severe urinary incontinence, but these systems are far less satisfactory for older men who are more likely to experience continence problems. For women continence management solutions are extremely limited. Internal catheters are associated with a host of medical problems, particularly urinary tract infections and urethral erosions. Large disposable pads to absorb urine are a safer option but are far from an ideal solution. Pads are bulky and require frequent changing if the wearer is to remain comfortable and free from odour problems. For immobile patients and those with sensory loss, wet pads can exacerbate the risk of pressure injury.

One of the major problems to consider when designing a device to cope with incontinence is the very high flow rate which is a characteristic of urine flow. During a void the urine flow rate increases rapidly to a peak rate of 25-30 millilitres per second. To pump urine directly requires a powerful pump able to reach a high flow rate as soon as it is activated. This is extremely difficult to achieve and would require a much larger pump and power source than can be easily transported.

WO 00/57784, the entire contents of which are incorporated herein by reference, discloses a urine collection device in which a vacuum is maintained. When urine is detected at an interface between the device and its user, a valve is opened allowing the drawing of urine into a storage container. In this device, a filter is provided in the lid of the container. The filter allows urine to pass through into the container, but resists the passage of air/gas. This has the effect of preserving the vacuum stored in the container.

The present invention seeks to provide improvements that may be used with the device disclosed in WO 00/57784. Natural urine has a high particulate load. For example, it may contain bacteria, epithelial cells, red and white blood cells, renal casts, uromucoid and albumin. Other matter, such as stray hairs, talcum powder and skin flakes could also be found in urine in the device. These particles may rapidly block the filter, which then needs to be replaced after each use. It is suggested that this problem could be reduced by using a filter having a large effective filter area, but this necessarily increases the size of the device, which is undesirable.

According to an aspect of the present invention, there is provided a urine collection device including: a container for storing collected urine; a first filter means at or adjacent an opening of the container, the filter means allowing egress of urine or other aqueous fluids into the container; the device including a second filter means arranged such that urine passes through the second filter means before passing through the first filter means, the second filter means having a higher capacity than the first filter means.

In this arrangement, the second filter means is able to filter out particulate material that could clog the first filter means. The second filter means thus protects the first filter means and extends the life of the first filter means such that it does not need to be replaced after each use. It is preferred that the second filter means could be used for half a day before replacement, more preferably for a whole day, more preferably for three or four days, even more preferably for a week.

By “having a higher capacity” we mean that the second filter means is able to hold a higher load of particulate material than the first filter means. The presence of the second filter means thus reduces the amount of particulate material reaching the first filter means by trapping such matter before it reaches the first filter means.

The second filter means preferably comprises a depth filter. This is a particularly suitable type of filter because it enables a large amount of particulate material to be trapped. The depth filter may be formed from glass fibre, for example.

Preferably, the second filter means has a nominal pore size smaller than that of the first filter means. This helps to reduce the amount of larger particles reaching the first filter means.

Preferably the second filter means has a pore size smaller than 8 μm. This enables the second filter means to filter out the most clogging types of particles.

In one embodiment, the second filter means and the first filter means are provided together within a single unit. This provides for a compact arrangement that does not substantially increase the overall size of the device. The filters in this arrangement are also simple for the user to replace.

In the preferred embodiment, the unit containing the first filter means and the second filter means comprises a lid for the container. This provides for a convenient location for the filter means that does not substantially increase the overall size of the device.

Preferably the unit is provided with an air outlet to allow air to be pumped out of the container. This allows the unit to provide also means for a vacuum to be applied to the container, enabling the container itself to be a simple receptacle.

In an alternative embodiment, the second filter means is provided in a unit separate from the first filter means, and is arranged upstream of the first filter means within the urine collection device. In this arrangement, the second filter means can easily be replaced without affecting the rest of the device.

According to a second aspect of the invention, there is provided an air outlet for a urine collection device comprising: a conduit for air passage for location between a urine storage container and a pumping means; wherein the conduit includes a material that allows passage of air but that does not allow passage of liquid and an odour-absorbing and/or moisture-absorbing material through which air passes when it is pumped out of the urine storage container by the pumping means.

The presence of a material that allows passage of air but that does not allow passage of liquid helps to protect the pumping means that has been connected to the air outlet from being damaged by residual moisture in the air. This may be further enhanced by the presence of moisture-absorbing material, which may remove moisture from air. Prevention of moisture reaching the pumping means helps to increase the life and efficiency of the pumping means. The provision of an odour-absorbing material helps to prevent any odour from the urine from escaping from the container. This enables the user to keep the device discreet.

Preferably the material that allows passage of air but that does not allow passage of liquid is a hydrophobic membrane through which air passes when it is pumped out of the urine storage container by the pumping means. A hydrophobic membrane can prevent aqueous liquid exiting the device. This helps to ensure that any air leaving the container is as dry as possible.

The conduit may include a second hydrophobic membrane. This acts as a back-up should the moisture-absorbing material become saturated thereby further protecting the pumping means.

In the preferred embodiment, the conduit includes a hydrophobic membrane located upstream of the odour-absorbing and/or moisture-absorbing material and a hydrophobic membrane located downstream of the odour-absorbing and/or moisture-absorbing material. The hydrophobic membrane located upstream of the odour-absorbing and/or moisture-absorbing material helps to prevent any urine from being sucked out of the container when a vacuum pump is connected to the air outlet. The hydrophobic membrane located upstream of the odour-absorbing and/or moisture-absorbing material helps to ensure that air leaving the container is as dry as possible, in order to protect any vacuum pump that may be attached to the air outlet. The hydrophobic membrane located downstream of the odour-absorbing and/or moisture-absorbing material acts as a back-up should the moisture-absorbing material become saturated.

The hydrophobic membrane may have a pore size configured to prevent passage of bacteria. This reduces the chance of contamination of the pumping means.

In a preferred embodiment the moisture-absorbing material is silica gel.

According to a third aspect of the present invention there is provided a filter assembly for a urine collection device comprising a pathway through which urine from a user can flow to a storage container, the pathway including an upstream filter means and a downstream filter means through which the urine must pass, wherein the upstream filter means has a higher capacity than the downstream filter means.

In this arrangement, the upstream filter means is able to filter out particulate material that could clog the downstream filter means. This protects the downstream filter means thereby extending its life such that it needs to be replaced less often. In the preferred embodiment, the downstream filter means is a membrane filter having a high bubble point.

Preferably the assembly further includes an air outlet to allow air to be pumped out of a storage container. This allows the assembly to provide also means for a vacuum to be applied to the container, enabling the container itself to be a simple receptacle.

In a preferred embodiment, the upstream filter means and the downstream filter means are provided within the same housing. This provides for a compact arrangement that does not substantially increase the overall size of the device.

Preferably the housing comprises a lid for a urine storage container. This provides for a convenient location for the filter means that does not substantially increase the overall size of the device.

Preferred embodiments of the present invention are described below, by way of example only, and with reference to the accompanying drawings, in which:

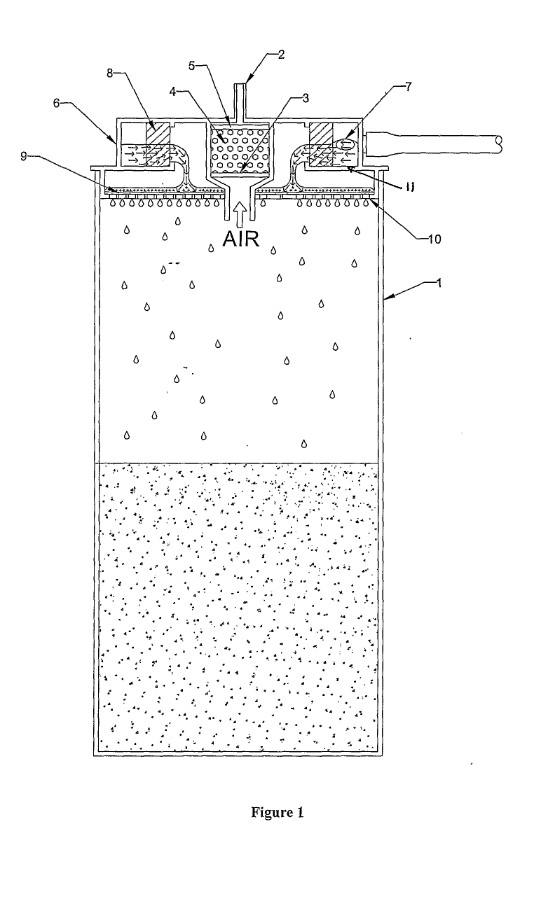

FIG. 1 shows a cross-section of a container and filter assembly according to an embodiment of the invention;

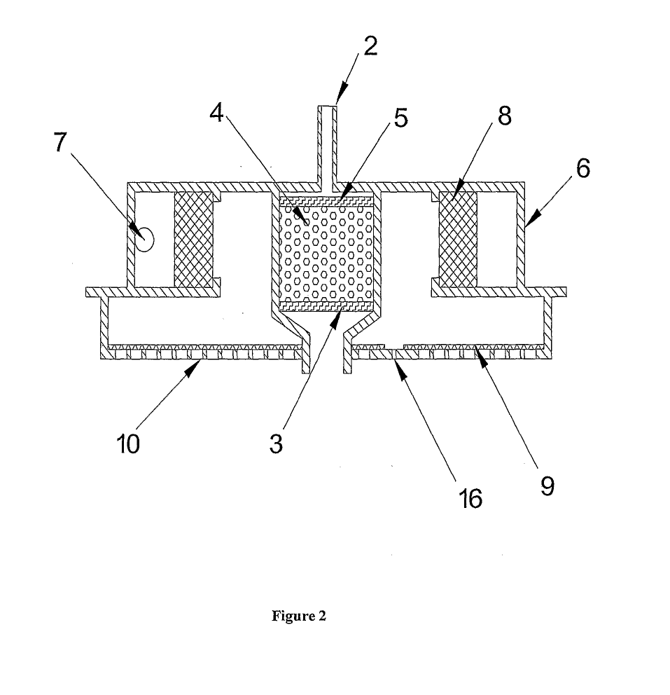

FIG. 2 shows an enlarged view of the filter assembly shown in FIG. 1;

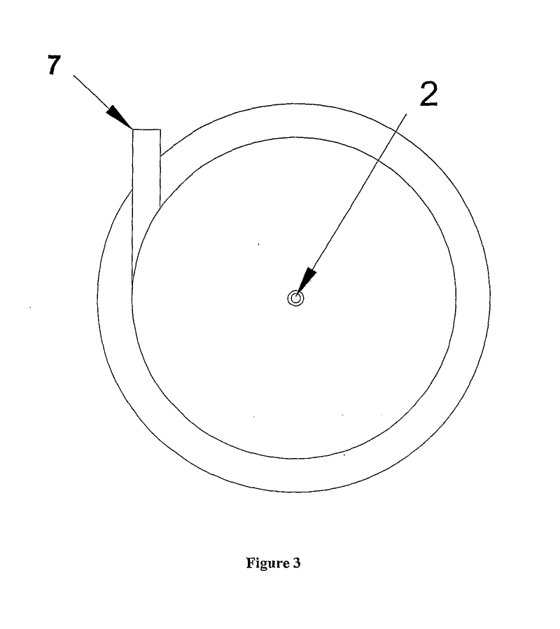

FIG. 3 shows a top view of the filter assemblies of FIG. 2; and

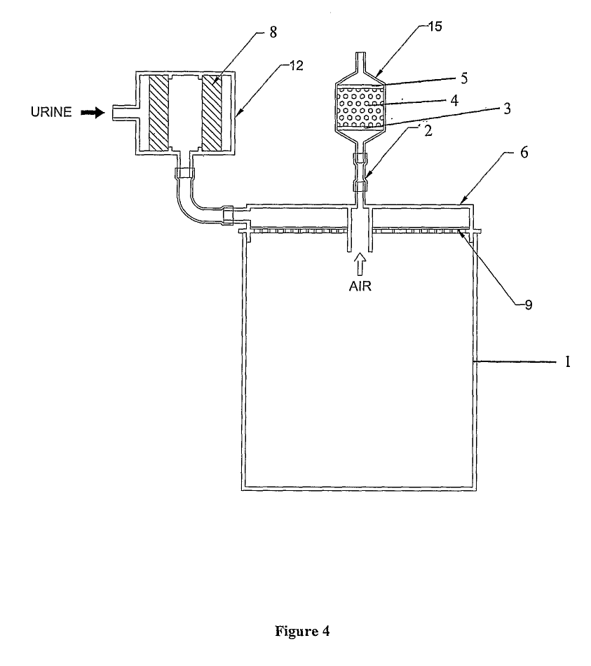

FIG. 4 shows a cross-section of a container and a filter according to an alternative embodiment of the invention.

Referring firstly to FIGS. 1 to 3, a preferred embodiment of the present invention includes a urine storage container 1 in which a vacuum may be maintained. The container is provided with a lid 6 that forms an air tight seal with the container 1. The lid 6 is comprised of impervious plastic moulding. Extending through the centre of the lid 6 is an air outlet 2. This allows air to be pumped out of the container, and is described in more detail below.

The base of the lid 6 comprises a perforated rigid filter support 10. A membrane filter 9 having a high bubble point is incorporated into the lid 6, and rests on the filter support 10. The membrane filter 9 allows urine to pass through into the container 1, whilst substantially preventing the passage of air. The membrane filter 9 could be a cellulose ester with a defined pore size between 0.8 and 8 μm, for example. This pore size has been found to be the maximum that prevents the passage of air and thus helps to maintain an ideal flow rate. The membrane filter is a high bubble point filter, which is impervious to air at the maximum pressure that the vacuum in the storage container reaches. Membrane filter 9 and filter support 10 include by-pass hole 16 (see FIG. 2), the purpose of which is explained below. The by-pass hole 16 has a diameter of 0.1-1 mm and is positioned towards the centre of the filter support 10 as shown in FIG. 2. The membrane filter 9 contains a corresponding hole of non-critical size.

Internal filter supports 11 extend radially inwardly towards the centre of the lid 6 and above membrane filter 9. Resting on the internal filter supports 11 is an annular pre-filter 8 comprising pleated glass fibre having an area, in this example, of at least 128 cm². The pre-filter 8 is located between the membrane filter 9 and the urine inlet port 7, and its nominal pore size is smaller than that of the membrane filter 9 (typically between 0.1 μm and 8 μm). The lid 6 thus surrounds both the membrane filter 9 and the pre-filter 8. This provides a compact arrangement that is simple for the user to replace when required.

The air outlet 2 extends through the lid 6, from the container 1 to a vacuum pump (not shown). Inside the air outlet 2 is provided a first hydrophobic filter 3, a layer of material that absorbs odour and moisture 4, such as activated carbon granules or silica gel, and then a second hydrophobic filter 5. These are arranged such that all air pumped out of the storage container 1, must pass through all of these layers. In preferred embodiments, the hydrophobic filters (3, 5) have a pore size small enough to prevent bacteria from passing through the filter (for example, 0.2 μm).

In use, a vacuum is maintained within the storage container 1 by means of a vacuum pump and valve as described in WO 00/57784. In the preferred embodiment of the present invention, when the presence of urine is detected at an interface between the device and its user, a solenoid valve opens to allow urine to be drawn into the container 1, by means of the vacuum previously set up. The urine flows into the lid 6 at the urine inlet port 7, from where it passes through the pre-filter 8. The pre-filter 8 removes any particulate matter from the urine.

However, the pre-filter 8 has sufficient area (for example, 128 cm²) and pore size to allow unrestricted flow of urine into the container 1. A suitable pore size could be 0.1 μm to 8 μm, for example. The filtered urine is then pumped through membrane filter 9, which prevents air from entering the container 1 thereby enabling the vacuum within the container 1 to be maintained. The urine can then be stored in the container until it is convenient for the user to empty it.

The vacuum within the container 1 is formed and maintained by pumping out air from the container 1 by means of a vacuum pump. A urine sensor, valve and pressure sensor operate to ensure the vacuum is maintained irrespective of whether or not urine is being produced. A detailed explanation of the operation of this system is given in WO 00/57784. In the preferred embodiment of the present invention, the air is pumped out via the air outlet 2, provided in the lid 6. The air passes through the first hydrophobic filter 3, which substantially prevents any of the collected urine from being sucked out. The air then passes through the odour- and moisture-absorbing material 4. This removes any residual moisture from the air and serves to ensure that the air being removed is as dry as possible. In this way, the vacuum pump is protected from any residual moisture in the air. Further, it serves to reduce any odour that may be present. Finally, the air passes through the second hydrophobic filter 5 which prevents excess moisture from the desiccant entering the pump.

The membrane filter 9 should be wetted before use. Once the membrane filter 9 has been wetted it is impervious to air at the maximum pressure that the system is set to produce. Therefore, both to remove the airlock and to allow the vacuum to reach the solenoid valve and ultimately the interface, the by-pass hole 16 is included in the filter support 10. The by-pass hole 16 allows the removal of the air lock at the beginning of second and subsequent urine flows. The membrane filter 9 will preferentially allow liquid rather than air to pass when a mixed stream is drawn through.

At a convenient moment, or when the container 1 is full, the user simply releases the vacuum applied to the container 1, removes the lid 6 containing the filters 8, 9 and the air outlet 2, and empties the container 1. The lid 6 can then be replaced and the vacuum reapplied to render the device ready for use.

There are various modifications that can be made to the above embodiment.

Membrane filter 9 could have a smaller pore size, such as between 0.8 and 8 μm. However, if a smaller pore size is used, a larger filter area is required.

The pre-filter 8 need not be formed from glass fibre, any high capacity filter material or depth filter could be used. A series of separate filters could be used as a pre-filter 8. These could have different pore sizes, for example, gradually decreasing in size. A membrane filter could even be used, but this would need to have quite a large filter area if it were not to clog up too quickly. It need not be annular; any arrangement that ensures the urine passes through the pre-filter 8 before passing through the membrane filter 9 could be used.

Other moisture- and odour-absorbing materials 4 can be used in the air outlet 2. For example, a mixture of carbon granules and silica gel.

A liner bag could be attached to the lid 6, to extend into the container 1. The liner bag would contain the urine and reduce the possibility of contamination of the container 1.

The by-pass hole 16 could be a 0.1-1 mm hole in the membrane filter 9 rather than in the filter support 10.

Instead of requiring removal of the lid 6 in order to enable the container 1 to be emptied, a tap can be included in the container 1 for urine removal. A suitable arrangement is described in WO 00/57784.

An alternative embodiment of the device is shown in FIG. 4. This embodiment also comprises a container 1 for storing collected urine and a lid 6 containing a membrane filter 9 and an air outlet 2. However, in this embodiment, the pre-filter 8 is provided within a filter housing 12 that is separate from lid 6, which houses the membrane filter 9. The pre-filter 8 is arranged upstream of membrane filter 9 and functions in the same way as described above for the embodiment of FIGS. 1 to 3.

In the embodiment of FIG. 4, two hydrophobic filters 3, 5 are provided sandwiching the moisture- and odour-absorbing material 4 within a separate air filtration cartridge 15 that attaches to air outlet 2. Again, the air outlet 2 and air filtration cartridge 15 function as described above for the embodiment of FIGS. 1 to 3.

An advantage of the embodiment of FIG. 4 is that once the capacity of the pre-filter 8 has been exceeded, the pre-filter 8 can be replaced without the need also to replace the membrane filter 9.

Other arrangements can be envisaged. For example, the lid could house the membrane filter 9 with the air outlet 2 containing the hydrophobic filters 3, 5 and moisture- and odour-absorbing material 4, the pre-filter 8 being housed in a separate unit 12. Alternatively, the lid 6 could house the membrane filter 9 with the pre-filter 8, the hydrophobic filters 3, 5 and the moisture- and odour-absorbing materials being housed within a separate unit, such as air filtration cartridge 15.

It will be clear to the skilled person that the modifications described with reference to the embodiment of FIGS. 1 to 3 are equally applicable to the embodiment of FIG. 4.

The disclosures in United Kingdom patent application no. GB 0520863.2, from which this application claims priority, and in the abstract accompanying this application are incorporated herein by reference.

1-20. (canceled)

21. A urine collection device including:
a. a container for storing collected urine;
b. a first filter at or adjacent an opening of the container, the first filter allowing egress of urine or other aqueous fluids into the container;
c. a second filter arranged such that urine passes through the second filter before passing through the first filter, the second filter having a higher capacity than the first filter.

22. The urine collection device of claim 21 wherein the second filter comprises a depth filter.

23. The urine collection device of claim 21 wherein the second filter comprises glass fibre as the filter material.

24. The urine collection device of claim 21 wherein the second filter has a nominal pore size smaller than that of the first filter.

25. The urine collection device of claim 21 wherein the second filter has a pore size smaller than 8 micrometers.

26. The urine collection device of claim 21 wherein the second filter and the first filter are provided together within a single unit.

27. The urine collection device of claim 26 wherein the unit containing the first filter and the second filter comprises a lid for the container.

28. The urine collection device of claim 21 wherein the second filter:
a. is provided in a unit separate from the first filter, and
b. is arranged upstream of the first filter within the urine collection device.

29. The urine collection device of claim 21 including an air outlet to allow air to be pumped out of the container, the air outlet comprising:
a. a conduit for air passage for location between a urine storage container and a pump, wherein the conduit includes a material that allows passage of air but that does not allow passage of liquid, and
b. an odour-absorbing and/or moisture-absorbing material through which air passes when it is pumped out of the urine storage container.

30. The urine collection device of claim 29 wherein the material is a hydrophobic membrane through which air passes when it is pumped out of the urine storage container.

31. The urine collection device of claim 30 wherein the hydrophobic membrane has a pore size configured to prevent passage of bacteria.

32. The urine collection device of claim 29 wherein the conduit includes:
a. a first hydrophobic membrane located upstream of the odour-absorbing and/or moisture absorbing material, and
b. a second hydrophobic membrane located downstream of the odour-absorbing and/or moisture absorbing material.

33. The urine collection device of claim 29 wherein the moisture-absorbing material is silica gel.

34. The urine collection device of claim 21 comprising a pathway through which urine from a user can flow to the container, the pathway including an upstream filter and a downstream filter through which the urine must pass, wherein the upstream filter has a higher capacity than the downstream filter.

35. A urine collection device including:
a. a container having a container opening leading to a container interior space for receiving urine;
b. a flow passage leading to the container interior space, the flow passage including:
(1) a first filter extending across at least a major portion of the container opening, wherein urine flows through the first filter into the container interior space,
(2) a second filter through which urine flows to enter the first filter, the second filter:
(a) having a higher capacity whereby it may hold a higher load of particulates than the first filter,
(b) extending about a path at least substantially surrounding a central area, with the second filter having an inner circumference adjacent the central area and an opposing outer circumference, wherein urine flows through the flow passage between the inner and outer circumferences.

36. The urine collection device of claim 35 wherein:
a. the second filter has a smaller pore size than the first filter; and
b. the volume of the second filter through which urine flows is substantially greater than the volume of the first filter through which urine flows.

37. The urine collection device of claim 35 wherein the first filter is situated within a lid removably fit in the container opening.

38. A urine collection device including:
a. a container having a container opening leading to a container interior space for receiving urine;
b. a lid removably fit in the container opening, the lid including:
(1) a first filter through which urine flows into the container interior space,
(2) a second filter through which urine within the lid flows to enter the first filter, the second filter having a higher capacity whereby it may hold a higher load of particulates than the first filter.

39. The urine collection device of claim 38 wherein:
a. the second filter extends about a path at least substantially surrounding a central area, with the second filter having an inner circumference adjacent the central area and an opposing outer circumference;
b. the first filter extends across at least a major portion of the container opening;
c. a urine inlet port supplies urine to the outer circumference of the second filter; and
d. at least some of the urine entering the urine inlet port flows from the outer circumference of the second filter to the inner circumference of the second filter, and subsequently to the first filter.

40. The urine collection device of claim 38 wherein an air outlet passage extends:
a. through the first filter, and
b. through the central area about which the second filter extends.
